(19) United States
(12) Patent Application Publication
(10) Pub. No.: US 2025/0176096 A1
(43) Pub. Date: May 29, 2025

(54) FLEXIBLE PLATE STRUCTURE FOR SMOKE-REDUCTION IN SURGICAL PROCEDURES

(71) Applicant: JOHNSON MEDTECH LLC, Vandalia, OH (US)

(72) Inventors: Libing ZHANG, Sharon, MA (US); Kenneth HEBERLING, Royal Oak, MI (US); Shane SNIPE, Troy, MI (US)

(21) Appl. No.: 18/439,637

(22) Filed: Feb. 12, 2024

Related U.S. Application Data
(63) Continuation of application No. PCT/US2021/045747, filed on Aug. 12, 2021.

(51) Int. Cl. H05K 1/02; A61B 18/00; A61B 18/14; B03C 3/45; B03C 3/64

(52) U.S. Cl. CPC H05K 1/0213; A61B 18/14; B03C 3/45; B03C 3/64; H05K 1/028; A61B 2018/00589; A61B 2018/00601; A61B 2218/008; B03C 2201/26; H05K 2201/10151

(57) ABSTRACT

Example approaches are directed to an apparatus for reducing smoke created by a surgical instrument in a procedure, e.g., as part of a surgical system. The apparatus may include an insulating vessel and a flexible printed circuit disposed within the vessel, with the vessel including an inlet and an outlet. The flexible printed circuit includes a substrate layer and a conductive layer disposed on the substrate. The conductive layer includes two charge conducting portions or electrodes having opposite electrical charges, which are spaced apart by an electrically insulated portion. The charge conducting portions are positioned between the inlet and outlet of the vessel such that particles entering the inlet are electrically charged and drawn to one of the charge conducting portions.

### CROSS REFERENCE TO RELATED APPLICATIONS

This non-provisional patent application is a continuation application of PCT Application No. PCT/US2021/045747, filed Aug. 12, 2021, which is incorporated herein by reference in their entirety.

### FIELD OF THE INVENTION

The invention relates to the technical field of valves, in particular to a multi-port valve and a thermal management system having the multi-port valve.

### BACKGROUND OF THE INVENTION

Electrosurgical device is used extensively nowadays for dissecting and coagulating targeted tissues. With these energized tools to perform minimally invasive procedures, smoke is produced during energized operation. One option is to remove the surgical instrument and wait for the smoke evacuation, or clear the field with other inert gases, before re-inflate/expand the operating field. US patent U.S. Pat. No. 10,722,294 discloses a surgical instrument with ion-generating electrode and a conductive portion to collect the smoke dust, which keeps the operation field clear for surgeon to see. In this configuration, precipitation uses only a negative electrode inserted into patient, the patient is placed on a positive plate. However, an undesirable side effect is that precipitation causes negatively-charged smoke particles to attract to the patient's tissues.

### SUMMARY OF THE INVENTION

In at least some example approaches, an apparatus for reducing smoke in a surgical procedure includes an insulating vessel and a flexible printed circuit disposed within the vessel. The insulating vessel includes an inlet and an outlet. The flexible printed circuit includes a substrate layer and a conductive layer disposed on the substrate. The conductive layer includes two charge conducting portions having opposite electrical charges, which are spaced apart by an electrically insulated portion. The charge conducting portions are positioned between the inlet and outlet of the vessel such that particles entering the inlet are electrically charged and drawn to one of the charge conducting portions.

In one or more example approaches, a surgical system includes a surgical instrument and an apparatus for reducing smoke created by the surgical instrument in a procedure. The apparatus includes an insulating vessel and a flexible printed circuit disposed within the vessel, with the vessel including an inlet and an outlet. The flexible printed circuit includes a substrate layer and a conductive layer disposed on the substrate. The conductive layer includes two charge conducting portions having opposite electrical charges, which are spaced apart by an electrically insulated portion. The charge conducting portions are positioned between the inlet and outlet of the vessel such that particles entering the inlet are electrically charged and drawn to one of the charge conducting portions.

In at least some example illustrations herein, a method of making an apparatus for reducing smoke in a surgical procedure includes printing a flexible printed circuit. The flexible printed circuit may include a substrate layer and a conductive layer disposed on the substrate. The conductive layer includes two charge conducting portions having opposite electrical charges and spaced apart by an electrically insulated portion. The method may further include positioning the flexible printed circuit within an insulating vessel having an inlet and an outlet, wherein the charge conducting portions are positioned between the inlet and outlet and configured to electrically charge particles entering the inlet such that the particles are drawn to one of the charge conducting portions.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows a perspective view of an apparatus for smoke reduction, e.g., in surgical applications, including a flexible printed circuit electrostatic precipitator, according to an example approach;

FIG. 2A shows a top view of the flexible printed circuit electrostatic precipitator of FIG. 1, with the flexible printed circuit electrostatic precipitator in a flattened or unrolled state, according to one example;

FIG. 2B shows a section view of the flexible printed circuit electrostatic precipitator of FIG. 2A taken through line 2B-2B, according to an example;

FIG. 3A shows an exploded perspective view of another electrostatic precipitator, according to one example approach;

FIG. 3B shows the electrostatic precipitator of FIG. 3A after assembly, according to one example;

FIG. 4 is a schematic illustration of a surgical system including an electrostatic precipitator, according to an example; and

FIG. 5 is a process flow diagram for an example method of manufacturing a smoke-reducing apparatus, according to an example illustration.

### DETAILED DESCRIPTION

Electrosurgical devices may be used for dissecting and coagulating targeted tissues during a surgical procedure. These energized tools may be used to perform minimally invasive procedures and may produce smoke when the tool is energized. The smoke may cloud the view of the surgical site and is undesirable. Generally, smoke must be cleared by removing the instrument and waiting for smoke to clear or be evacuated to allow the procedure to continue. Various smoke-clearing and smoke-reducing devices have been developed. In one example, a surgical instrument includes uses a negatively charged electrode that is inserted into patient to allow smoke particles to become negatively charged. The smoke particles subsequently accumulate according to placement of a positively charged plate, upon which the patient is positioned. However, the negatively charged smoke particles may also become attracted to the patient's tissues. This causes at least some of the smoke particles to become attached to the patient and may result in the particles being left inside the patient. Further, the system is generally bulky and has relatively complex wiring and electrical connections. As a result, any movement that may loosen a connection, as may occur during the procedure, may reduce effectiveness of the device. The positioning of the negative electrode within the patient is also undesirable, particularly where a high voltage potential is employed, due to additional patient protection that may be necessary to prevent the potential from coming into direct contact with the patient.

Accordingly, in example approaches herein an electrostatic precipitator or apparatus is provided, which includes positive and negative charge conducting portions that may be utilized in a position external to the patient. The possibility of a patient coming into contact with the electrical potential used during the procedure is therefore substantially eliminated. Additionally, providing the charge conduction portions or electrodes at a position external to the patient may also facilitate the use of flexible structures and materials. For example, charge conduction portions may be provided by way of a relatively thin, flexible circuit. In some approaches, relatively low-cost, printed electronics (PE) materials, e.g., carbon or silver inks, may be used to provide electrical communication or circuits for the device. Alternatively, a flexible printed circuit (FPC) having copper and gold materials may be used. Flexible circuits may allow reduced connection points, as an electrical connection for the device is generally needed only between the flexible circuit and a power supply. In example approaches using an electrostatic precipitator mounted external to the patient, size limitations typical of patient-internal applications also are generally removed. Accordingly, relatively larger charge conducting devices or electrodes may be used, relatively increasing capacity for capturing particles during a surgical procedure. The positive and negative charge conducting portions or electrodes may also be provided with any gap distance between that is convenient. Generally, flexible printed circuits (FPC) are relatively thin in thickness and are flexible to bend and conform, e.g., to allow shaping or fitting within an insulative vessel. Additionally, the dimensions of various electrostatic precipitators disclosed herein, e.g., copper based FPCs or Printed Electronics (PE) based electrostatic precipitators, can be easily designed to accommodate space constraints, and to meet specific surgical functions depending on surgical tool type sand different operation applications.

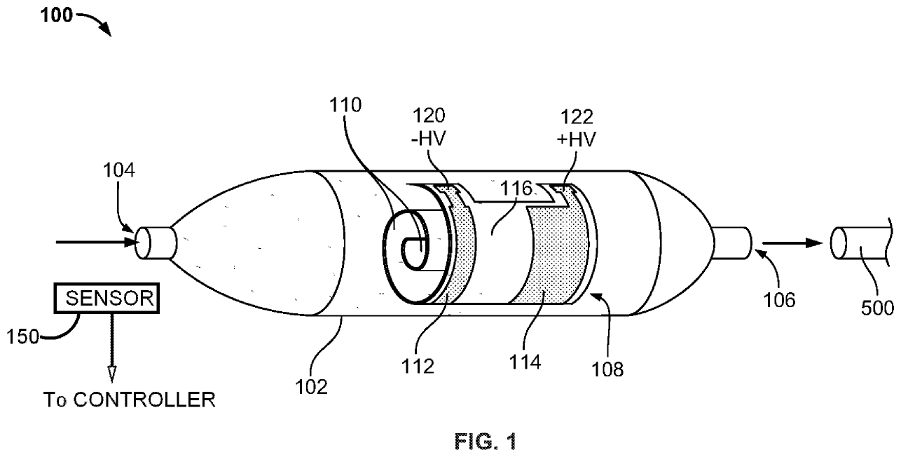

Referring now to FIG. 1, an illustrative apparatus 100 for smoke reduction, e.g., in surgical applications, is shown. The apparatus 100 includes a vessel 102 having an inlet 104 and an outlet 106. As will be described further below, the inlet 104 may be positioned near or adjacent a surgical site of a patient. Smoke, soot, debris, or other particles, e.g., caused by a surgical tool, may be drawn into the vessel 102 via the inlet 104. The smoke, particles, soot, etc. may be collected or scrubbed, with air exiting the vessel 102 via the outlet 106. As seen in FIG. 1, the vessel 102 may have a generally cylindrical body extending between the inlet 104 and the outlet 106. The vessel 102 may be formed in any manner or method that is convenient, e.g., molding or blow molding. The vessel 102 may also be formed in separate parts, e.g., one part having the inlet 104 and a second part having the outlet 106, which are assembled around components internal to the vessel 102, e.g., an electrostatic precipitator 108. The vessel 102 may be formed of an insulating material that generally provides a barrier between a patient and electrical components within the vessel 102, e.g., the electrostatic precipitator 108. The vessel 102 may be a molded plastic or other material having insulative properties or dielectric performance, thereby avoiding arcing of electricity from internal components, e.g., electrostatic precipitator 108, to an external surface of the vessel 102. The inlet 104 and/or outlet 106 may narrow from the cylindrical body of the vessel 102 to match tubing or hoses to connect the inlet 104 and/or outlet 106 of the apparatus 100 with airflow. For example, airflow from the surgical site may be drawn in via tubing or hoses to the inlet 104. Similarly, airflow from the apparatus 100 may be conducted from the outlet 106 of the vessel 102 via tubing 500.

The electrostatic precipitator 108 may be positioned within the vessel 102 in any manner that is convenient. Merely by way of example, one or more supports (not shown in FIG. 1) may extend from internal surfaces of the vessel 102 to suspend the electrostatic precipitator 108 within the vessel 102. The electrostatic precipitator 108 may be configured to remove or collect smoke, particles, soot, or the like from air or fluid drawn into the inlet 104. In the example illustration of FIG. 1, the electrostatic precipitator 108 is a flexible printed circuit structure. As will be described in further detail below, the flexible printed circuit structure may be overlaid upon a substrate layer 110 in a generally flat configuration. The flexible printed circuit structure may subsequently be rolled to form a generally cylindrical shape or round cross-section to fit within cylindrical body of the vessel 102. The electrostatic precipitator 108 includes first and second charge conducting portions 112 and 114, respectively. The charge conducting portions 112, 114 have opposite electrical charges with respect to each other. In the example illustrated in FIG. 1, the first charge conducting portion 112 positioned adjacent the inlet 104 maintains a negative charge, while the second charge conducting portion 114 carries a positive charge. However, in some example approaches this orientation may be reversed. The first and second charge conducting portions 112, 114 are spaced apart by an electrically insulated portion 116. Generally, the charge conducting portions 112 and 114 are positioned between the inlet 104 and outlet 106 such that particles entering the inlet 104 are electrically charged by the charge conducting portion 112, and thus drawn to the charge conducting portion 114. The charge conducting portion 112 may be positioned at any distance with respect to the inlet 104 that is convenient. In the example illustrated in FIG. 1 where the first charge conducting portion 112 carries a negative charge, soot or smoke particles drawn into the inlet 104 pass near the negative charge conducting portion 112, thereby becoming negatively charged. As the particles pass further into the vessel 102 away from the inlet 104, the particles are drawn to the positive charge conducting portion 114 and may become trapped due to the persistent positive charge maintained by the positive charge conducting portion 114. The cleaned/scrubbed air (or other suitable fluid medium) may be drawn through the outlet 106 to a hose 500, which may be affixed to the outlet 106. In some examples it may be possible to clean the apparatus 100 and/or the electrostatic precipitator 108 to remove collected particles from the charge conducting portion 114. However, it is contemplated that the apparatus 100 and electrostatic precipitator 108 may be made from relatively inexpensive or disposable materials/components such as those disclosed herein, and thus may be discarded and/or replaced when a capacity of the apparatus 100 for collecting particles is reached.

In example illustrations herein, an electrical potential may be applied to the charge conducting portions 112, 114 via respective electrical inputs 120, 122. In some examples, a high-voltage electrical potential may be employed. More specifically, a high-voltage power supply may provide a direct current (DC) input to the electrostatic precipitator 108. Merely by way of example, an input voltage of 1000 Volts (V) to 50000 V DC may be used. In another example, a battery having a 12 V DC output and 3000 milliamp Hour (mAh) capacity may be employed to power the apparatus 100. The use of a battery for powering the apparatus 100 may be beneficial to the extent it may facilitate fewer connections of the apparatus 100 to a surgical system and allow easier disposal of the apparatus 100. Alternatively, the apparatus 100 may be directly plugged into a power source to create a desired voltage difference applied to the charge conducting portions 112 and 114. Generally, a greater electrical potential may result in greater effectiveness at retention of the particles to the electrostatic precipitator 108. It should be noted that a relatively higher voltage may also require a relatively larger gap G between the charge conducting portions 112 and 114 to reduce the possibility of electrical arcing between the charge conducting portions 112 and 114. The voltage potential is preferably configured to provide high enough voltage to charge the charge conducting portions 112, 114, but not so great as to create arcing between the charge conducting portions 112, 114. The gap G may also be determined based at least in part upon a creepage distance between the charge conducting portions 112, 114 across the insulated portion 116.

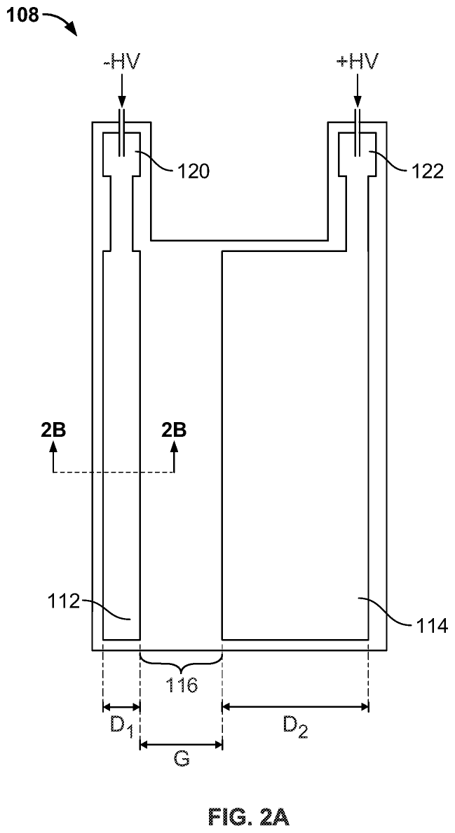

Turning now to FIGS. 2A and 2B, the example electrostatic precipitator 108 is illustrated and described in further detail. A top view of the electrostatic precipitator 108 is shown in a flattened or unrolled state in FIG. 2A, with a section view of a portion of the electrostatic precipitator 108 being shown in FIG. 2B. Charge conducting portions 112 and 114 of the electrostatic precipitator 108 may be provided in a conductive layer 118 that is disposed on the substrate layer 110. The conductive layer 118 is illustrated being secured to the substrate layer 110 with an adhesive layer 124, however in some approaches the conductive layer 118 may be directly applied to the substrate layer 100, e.g., where the conductive layer 118 is printed upon the substrate layer 110. The conductive layer 118 may be configured to receive a high-voltage electrical input to generate the opposite electrical charges for the charge conducting portions 112 and 114. For example, as seen in FIG. 2A, an input 120 may conduct a negative electrical potential to the charge conduction portion 112, while the input 122 conducts a positive electrical potential to the charge conducting portion 114. As noted above, the orientations of the positive and electrical inputs may be reversed in some examples.

Referring now to FIG. 2B, in some example approaches the electrostatic precipitator 108 includes a plurality of relatively thin layers overlaid upon each other. A base or substrate layer, electrical conductors, dielectric materials to protect the traces, and surface finish(es) on the exposed traces/plates/charge conducting portions may be provided in a generally layered structure, for example.

In the example electrostatic precipitator 108 illustrated in FIG. 2B, the conductive layer 118 may be printed or overlaid upon the substrate layer 110 in any manner that is convenient. Merely as example, the substrate layer 110 may include one or more plastic films, e.g., polyester, polyimide, polycarbonate, polyethylene terephthalate (PET), or the like. A thickness of the substrate layer 110 may be, for example, 12.5 microns to 250 microns. The substrate layer 110 is generally flexible such it may be wrapped, coiled, or otherwise shaped to facilitate fitting the electrostatic precipitator 108 into the vessel 102. The other layers, e.g., conductive layer 118, may also be flexible to allow shaping of the electrostatic precipitator 108.

The conductive layer 118 may be overlaid upon the substrate layer 110 with an adhesive layer 124, or as noted above may be directly applied to the substrate layer 110, e.g., in a printing process. The conductive layer 118 may form the inputs 120, 122 and their respective charge conducting portions 112, 114, as well as electrical traces leading between the input/charge conducting portion. The conductive layer 118 may be formed of any conductive material that is convenient, such as a copper material or a printed conductive ink. For example, the conductive layer 118 may be a screen printable ink, e.g., carbon or silver. In another example, the conductive layer 118 may be an etched copper circuit using flexible printed circuitry (FPC) technology.

An insulating or dielectric layer 126 may be overlaid upon the conductive layer 118, e.g., with another adhesive layer 128. In one example the insulating layer 126 is a printed solder mask, polymer or dielectric ink, or a polyester or polyimide film or laminate. The insulating layer 126 may be applied over the electrical traces and components of the conductive layer 118 to provide electrical isolation of the conductive layer 118.

Where the conductive layer 118 includes metallic materials, it may be beneficial to cover exposed portions, e.g., along the charge conducting portion 112 (and, for that matter, the charge conducting portion 114) with a surface finish or surface layer 130. Merely as examples, in an FPC circuit having metal conductors, a surface layer 130 may be applied in the form of an electroless nickel immersion gold, a gold (Au) plating, or the like. The gold surface layer 130 may generally provide an inert conductive surface finish, e.g., on the charge conducting portion 112. Additionally, a gold-based finish is biocompatible. A surface layer 130 may not be necessary for applications where an ink-based conductor is employed, as ink-based conductors are generally biocompatible and may not be prone to oxidation.

The electrostatic precipitator 108 may be formed with charge conducting portions 112, 114 of any size, shape, and configuration that is convenient. As best seen in FIG. 2A, the charge conducting portion 112 may have a width D₁, while the charge conducting portion 114 has a relatively larger width D₂. When the electrostatic precipitator 108 is rolled and positioned within the vessel 102 as illustrated in FIG. 1, each of the D₁ and D₂ dimensions may extend in a direction parallel to a direction of flow within the vessel 102, i.e., from the inlet 104 to the outlet 106. A gap G also separates the charge conducting portions 112, 114 in the same direction as the extent of the widths D₁ and D₂.

Performance of the electrostatic precipitator 108 may be modified by varying, among other parameters, the widths D1 and D2, the gap G, and voltage potential applied to the charge conducting portions 112 and 114. Generally, higher voltage potentials will impart electrical charges to particles with more effectiveness, resulting in a relatively greater share of particles flowing through the vessel 102 being removed from the flow through the vessel 102. Additionally, a larger width D₂ of the charge conducting portion 114 may facilitate a greater capacity for collecting particles by creating a greater surface area to which charged particles can adhere. On the other hand, it may be relatively less important for the charge conducting portion 112 to have a larger width or surface area, as particles need not adhere to the charge conduction portion 112. Rather, the charge conducting portion 112 need only be large enough and positioned in the vessel 102 so that as many particle entering the inlet 104 pass near enough to the charge conduction portion 112 to become charged, e.g., with a negative charge, causing them to be drawn to the charge conduction portion 114. As noted above, the gap G between the charge conducting portions 112 and 114 is preferably large enough to avoid electrical arcing between the charge conducting portions 112 and 114. At the same time, the gap G is preferably small enough so that the conducting portion 114 is close enough to attract the negatively charged particles sufficiently before they discharge. The voltage potential applied to the charge conducting portions 112 and 114 will also positively affect the ability of the apparatus 100 to attract and retain particles, i.e., a larger voltage potential will increase performance. At the same time, a larger voltage potential will result in a relatively greater risk of arcing between the charge conducting portions 112 and 114. Accordingly, a gap G and voltage potential for a specific application may be determined based upon a balancing of these and other factors.

The electrostatic precipitator 108 may be initially formed in a flat laminated sheet as illustrated in FIGS. 2A and 2B. The electrostatic precipitator 108 may be subsequently rolled into a cylindrical or coil shape to allow the electrostatic precipitator 108 to be fit into a vessel or other container, e.g., vessel 102.

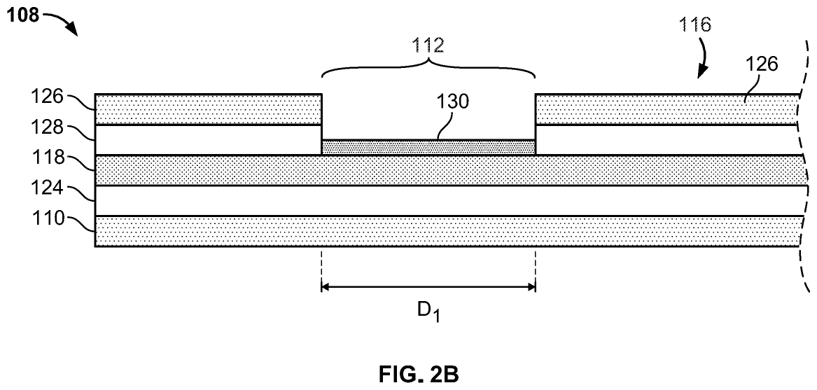

Turning now to FIGS. 3A and 3B, another example electrostatic precipitator 108′ is illustrated and described in further detail. The electrostatic precipitator 108′ employs a stacked construction of components including a first charge conducting portion 112′ and a second charge conducting portion 114′. The charge conducting portions 112′ and 114′ may be formed in any method that is convenient, e.g., a three-dimensional printing process, molding, etc. The charge conducting portions 112′ and 114′ are spaced apart by an insulating portion 136. End caps 132 and 134 are positioned at respective ends and are clamped together by a threaded bolt 138 which engages a nut 140. The electrostatic precipitator 108′ may be utilized in a vessel, e.g., vessel 102, and operated as described above regarding the electrostatic precipitator 108. More specifically, a voltage difference may be applied to the charge conducting portions 112′ and 114′, e.g., such that a negative charge is created at the charge conducting portion 112′ and a positive charge is created at the charge conducting portion 114′. Accordingly, particles entering a vessel containing the electrostatic precipitator 108′ become negatively charged as they pass near the charge conducting portion 112′. As the particles flow further through the vessel, they accumulate on the charge conducting portion 114′.

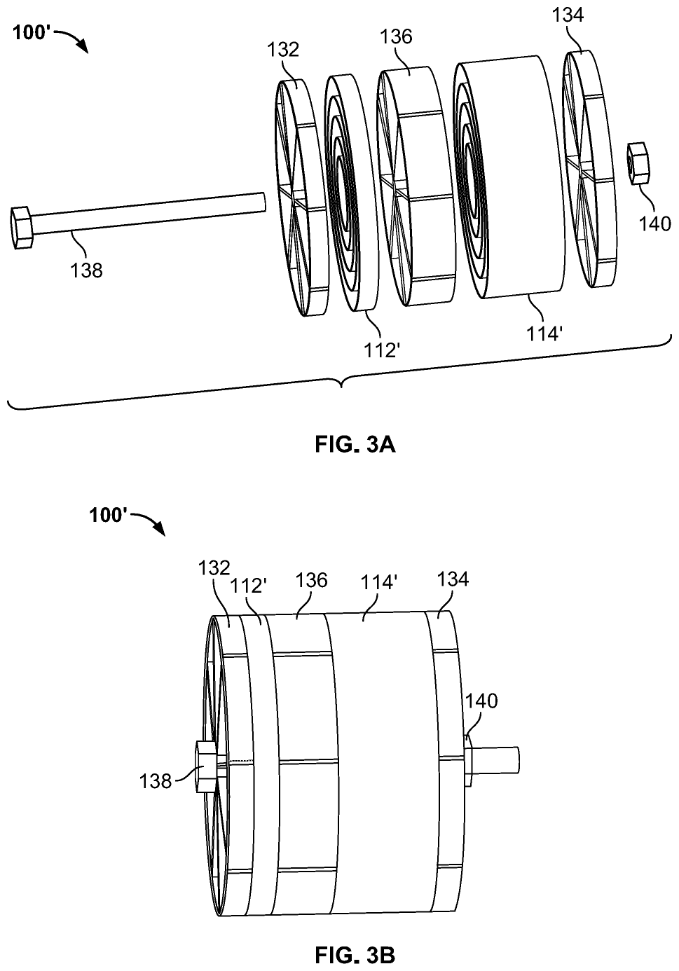

Turning now to FIG. 4, an example surgical system 1000 is illustrated. The system 1000 may include the above-described apparatus 100, which is positioned near a surgical site or patient P. A surgical instrument 200, e.g., a trocar or other tool, may be used during a surgical procedure to cut or ablate tissue, for example. In any case, the instrument 200 may cause smoke, soot, or other particles to propagate from the patient P. The apparatus 100 may be used to draw air away from the patient P, such that smoke, soot, or other particles are drawn into the apparatus 100. Particles entrained in the incoming airflow to the apparatus 100 are collected and retained within the apparatus 100. Airflow may continue from the apparatus 100 via hose 500, which may lead to a pump 300.

The pump 300 and apparatus may be actuated by a controller 400. The controller 400 may include a processor and a memory. The processor of the controller 400 may include any suitable processing equipment such as a central processing unit having single core or dual core, bus, logic circuitry, integrated circuitry, digital signal processor, graphics processor, any other suitable components for reading and executing computer instructions, or any combination thereof. The memory of the controller 400 may include any suitable computer readable storage medium or storage device such as, for example, a volatile memory, a non-volatile memory, a removable storage device, a solid state storage device, an optical device, a magnetic device, any other suitable component for storing and recalling information, or any combination thereof. The controller 400 may be configured to actuate the apparatus 100 in response to a detection of smoke, soot, or other particles being created by the surgical instrument 200. For example, the apparatus 100 may have a sensor 150, e.g., positioned adjacent inlet 104 (not shown in FIG. 4), that is configured to detect a presence of soot, smoke, or other particles such as may be created by the surgical instrument 200. The sensor 150 may be in communication with the controller 400, and accordingly the controller 400 may actuate the apparatus 100 when soot, smoke, or other particles are created by the surgical instrument 200. The sensor 150 may be an inductive sensor to sense an electrical current in the instrument 200, or a microphone to sense ambient noise or sound indicative of operation of the instrument 200, or a sensor to sense the air particulates. A sensor circuit may be used to detect a change in impedance as smoke particles are precipitated (or, for that matter, an absence thereof). In another example, the apparatus 100 is actuated periodically during a surgical procedure, e.g., after expiration of a timer or other device for periodically activating the apparatus 100. Alternatively, the precipitator 108 could be periodically activated, for example with a pre-programmed pulse waveform. The apparatus 100 may be actuated by connecting an input voltage with the charge conducting portions 112 and 114, by actuating a pump to draw an airflow into the vessel 102, or both.

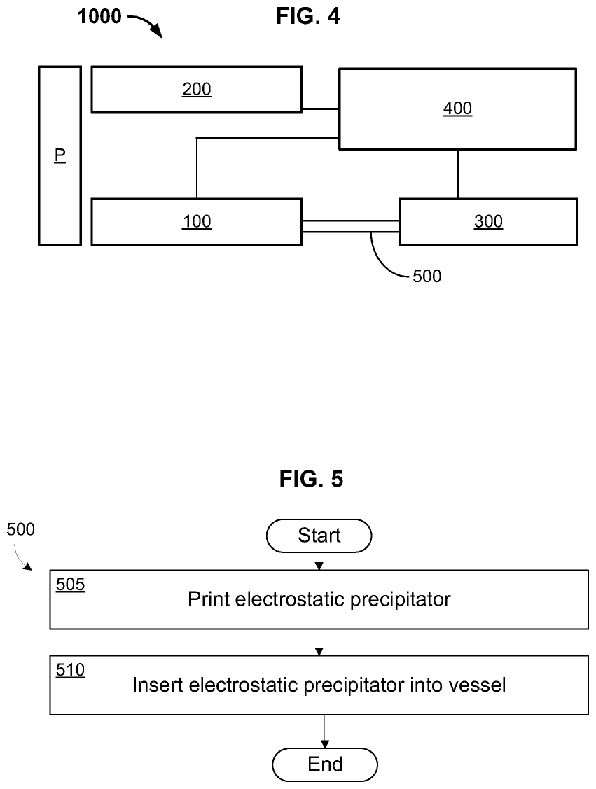

Turning now to FIG. 5, an example process 500 of manufacturing a smoke-reducing apparatus is described in further detail.

Process 500 may begin at block 505, where a flexible printed circuit is formed, e.g., via printing. For example, as noted above an electrostatic precipitator 108 may be formed by printing a conductive layer 118 upon a substrate layer 110. The conductive layer 118 may include charge conducting portions 112, 114 having opposite electrical charges that are spaced apart by an electrically insulated portion 116. As noted above, the substrate layer 110 may be formed of a flexible material to facilitate forming or shaping the electrostatic precipitator 108 into a vessel. Process 500 may then proceed to block 510.

At block 510, the flexible printed circuit may be positioned within an insulating vessel having an inlet and an outlet. For example, as discussed above an electrostatic precipitator 108 may be rolled to fit within a cylindrical vessel 102. Charge conducting portions of the electrostatic precipitator 108 may be positioned the inlet 104 and outlet 106 and may be configured to electrically charge particles entering the inlet 104 such that the particles are drawn to one of the charge conducting portions. For example, the first charge conducting portion 112 may have a negative charge that tends to cause particles flowing into the inlet 104 to become negatively charged, and drawn to the second charge conducting portion 114, which has a positive electrical charge.

Referring again to FIGS. 1, 2A, and 2B, the illustrated apparatus 100 may reduce airborne particle concentration in an environment and may include an electrically insulating vessel 102 having an inlet 104 and an outlet 106. The apparatus 100 may also include a flexible printed circuit 108 disposed within the vessel 102. The flexible printed circuit 108 may include a substrate layer 110 and a pair of electrodes, e.g., the charge conducting portions 112 and 114, disposed on the substrate 110. The electrodes 112, 114 may have, in use, opposite polarities and may be spaced apart by an electrically insulating portion 116. The electrodes 112, 114 may be positioned between the inlet 104 and outlet 106 such that particles entering the inlet 14 are electrically charged and drawn to one of the electrodes 112 or 114.

In at least some examples, the electrodes 112, 114 are configured to receive a high-voltage electrical input to generate the opposite polarities.

In at least some example approaches, the substrate layer 110 is formed of a flexible material.

In at least some example illustrations, the flexible printed circuit 108 includes a dielectric layer 126 covering a portion of a conductive layer 118, e.g., in which the electrodes 112 and/or 114 are provided. In at least some of these examples, the dielectric layer 126 includes one of a printed polymer, a printed dielectric ink, or a laminate polyimide.

In at least some examples, the insulating vessel 102 includes a cylindrical body between the inlet 104 and the outlet 106. In at least some of these example approaches, the flexible printed circuit 108 is rolled to fit within the cylindrical body of the vessel 102.

In at least some example approaches, the electrodes 112 and/or 114 are formed of a copper material or a printed conductive ink.

In at least some example illustrations, the apparatus 100 comprises a surface layer 130 overlaying the electrodes 112 and/or 114 to prevent oxidation of the electrodes 112 and/or 114.

In at least some examples, the apparatus 100 may include or a sensor configured to actuate the flexible printed circuit 108 in response to a detection of an actuation of a surgical instrument or a presence of the particles.

In at least some example approaches, the flexible printed circuit 108 is configured to periodically activate the electrodes 112, 114 during a surgical procedure.

In at least some example illustrations, the two electrodes 112, 114 comprise a negative electrode 112 and a positive electrode 114, with the negative electrode 112 positioned such that the negative electrode 112 applies a negative electrical charge to particles entering the inlet 104, and with the positive electrode 114 positioned between the negative electrode 112 and the outlet 106.

The example apparatuses 100 described above may be employed, e.g., in a surgical procedure, to reduce particle concentration in an environment, or to reduce smoke, soot, or other debris. The apparatuses 100 described above may be used in connection with the system 1000 and/or process 500, merely as examples. The example illustrations of an apparatus 100 advantageously provide an electrostatic precipitator device which remains insulated from a patient, e.g., by containing the electrostatic precipitator within a vessel. The apparatus 100 and/or the electrostatic precipitator 108 may also be flexible to fit within a filter module, to which air is circulated from within the body cavity through the apparatus 100, and back to the body cavity. The apparatus 100 may be connected to an external power source or may include either a battery or a dedicated power supply to provide a stand-alone unit for dedicated electrostatic precipitation in a disposable package. As the smoke precipitation is generally only needed while electrosurgery is being performed and immediately after, it may be advantageous to add a sensor to sense the operation so as to conserve power when necessary, as noted above.

The foregoing description includes exemplary embodiments in accordance with the present disclosure. These examples are provided for purposes of illustration only, and not for purposes of limitation. It will be understood that the present disclosure may be implemented in forms different from those explicitly described and depicted herein and that various modifications, optimizations, and variations may be implemented by a person of ordinary skill in the present art, consistent with the following claims.

What is claimed is:

1. An apparatus for reducing smoke in a surgical procedure, comprising:
an insulating vessel having an inlet and an outlet;
a flexible printed circuit disposed within the vessel, the flexible printed circuit comprising:
a substrate layer; and
a conductive layer disposed on the substrate, the conductive layer including a two charge conducting portions having opposite electrical charges and spaced apart by an electrically insulated portion, the charge conducting portions positioned between the inlet and outlet such that particles entering the inlet are electrically charged and drawn to one of the charge conducting portions,
wherein the apparatus further comprises a sensor configured to actuate the flexible printed circuit in response to one of a detection of an actuation of a surgical instrument or a presence of the particles.

2. The apparatus of claim 1, wherein the conductive layer is configured to receive a high-voltage electrical input to generate the opposite electrical charges.

3. The apparatus of claim 1, wherein the substrate layer is formed of a flexible material.

4. The apparatus of claim 1, wherein the flexible printed circuit includes a dielectric layer covering a portion of the conductive layer.

5. The apparatus of claim 4, wherein the dielectric layer includes one of a printed polymer, a printed dielectric ink, or a laminate polyimide.

6. The apparatus of claim 1, wherein the insulating vessel includes a cylindrical body between the inlet and the outlet.

7. The apparatus of claim 6, wherein the flexible printed circuit is rolled to fit within the cylindrical body of the vessel.

8. The apparatus of claim 1, wherein the conductive layer is formed of one of a copper material or a printed conductive ink.

9. The apparatus of claim 1, further comprising a surface layer overlaying the charge conducting portions to prevent oxidation of the charge conducting portions.

10. The apparatus of claim 1, wherein the flexible printed circuit is configured to periodically activate the negative charge conducting portion and the positive charge conducting portion during the procedure.

11. The apparatus of claim 1, wherein the two charge conducting portions comprise a negative charge conducting portion and a positive charge conducting portion, the negative charge conducting portion positioned such that the negative charge conducting portion applies a negative electrical charge to particles entering the inlet, the positive charge conducting portion positioned between the negative charge conducting portion and the outlet.

12. A surgical system, comprising:
a surgical instrument; and
an apparatus for reducing smoke created by the surgical instrument in a procedure, the apparatus comprising:
an insulating vessel having an inlet and an outlet;
a flexible printed circuit disposed within the vessel, the flexible printed circuit comprising:
a substrate layer; and
a conductive layer disposed on the substrate, the conductive layer including a two charge conducting portions having opposite electrical charges and spaced apart by an electrically insulated portion, the charge conducting portions positioned between the inlet and outlet such that particles entering the inlet are electrically charged and drawn to one of the charge conducting portion,
wherein the system further comprises a sensor configured to actuate the flexible printed circuit in response to one of a detection of an actuation of a surgical instrument or a presence of the particles.

13. The system of claim 12, wherein the flexible printed circuit is configured to periodically activate the charge conducting portions during the procedure.

14. A method of making an apparatus for reducing smoke in a surgical procedure, comprising:
printing a flexible printed circuit, the flexible printed circuit including a substrate layer and a conductive layer disposed on the substrate, the conductive layer including two charge conducting portions having opposite electrical charges and spaced apart by an electrically insulated portion; and
positioning the flexible printed circuit within an insulating vessel having an inlet and an outlet, wherein the charge conducting portions are positioned between the inlet and outlet and configured to electrically charge particles entering the inlet such that the particles are drawn to one of the charge conducting portions.

15. The method of claim 14, wherein the substrate layer is formed of a flexible material.

16. The method of claim 14, wherein the flexible printed circuit includes a dielectric layer covering a portion of the conductive layer.

17. The method of claim 14, wherein the insulating vessel includes a cylindrical body between the inlet and the outlet; wherein the method comprises rolling the flexible printed circuit to fit the flexible printed circuit within the cylindrical body of the vessel.

18. The method of claim 14, wherein the conductive layer is formed of one of a copper material or a printed conductive ink.
